(19) United States
(12) Patent Application Publication
(10) Pub. No.: US 2026/0213019 A1
(43) Pub. Date: Jul. 23, 2026

(54) EXPLAINABLE MULTI-MODAL TIME-SERIES PREDICTION FOR MEDICAL DECISION MAKING

(71) Applicant: NEC Laboratories America, Inc., Princeton, NJ (US)

(72) Inventors: Wenchao Yu, Plainsboro, NJ (US); Wei Cheng, Princeton Junction, NJ (US); Haifeng Chen, West Windsor, NJ (US); Yushan Jiang, Vernon, CT (US)

(21) Appl. No.: 19/319,123

(22) Filed: Sep. 4, 2025

Related U.S. Application Data
(60) Provisional application No. 63/692,754, filed on Sep. 10, 2024.
(60) Provisional application No. 63/747,405, filed on Jan. 21, 2025.

(51) Int. Cl. G16H 50/30; G06F 18/25

(52) U.S. Cl. CPC G16H 50/30; G06F 18/25

(57) ABSTRACT

Methods and systems include inferring a first prediction and an explanation based on a time series input and a text input using a multi-modal prototype-based encoder. A second prediction is inferred based on the text input and the explanation using a large language model. The first prediction and the second prediction are fused to generate a fused prediction. A reflection is generated based on the fused prediction, the second prediction, and the text input. The text input is refined based on the reflection.

### RELATED APPLICATION INFORMATION

This application claims priority to U.S. Application No. 63/692,754, filed on Sep. 10, 2024, and to U.S. Application No. 63/747,405, filed on Jan. 21, 2025, each incorporated herein by reference in its entirety.

### BACKGROUND

### Technical Field

The present invention relates to time series prediction and, more particularly, to time series prediction with large language models (LLMs).

### Description of the Related Art

Time series information is collected in a variety of scenarios, for example where sensors and services report measurements or events associated with particular timestamps. A multivariate time series may track many such measurements along a common time axis, and time series representing temporal variable dynamics can be applied across a diverse range of domains, such as healthcare, traffic, and energy.

Time series analysis based on deep learning helps to comprehend real-world system behaviors. However, time series may be characterized by their complex dependencies and evolving natures, which presents an analytical challenge for learning models. Time series may exhibit multi-mode behaviors over time.

### SUMMARY

A method includes inferring a first prediction and an explanation based on a time series input and a text input using a multi-modal prototype-based encoder. A second prediction is inferred based on the text input and the explanation using a large language model. The first prediction and the second prediction are fused to generate a fused prediction. A reflection is generated based on the fused prediction, the second prediction, and the text input. The text input is refined based on the reflection.

A system includes a hardware processor and a memory that stores a computer program. When executed by the hardware processor, the computer program causes the hardware processor to infer a first prediction and an explanation based on a time series input and a text input using a multi-modal prototype-based encoder, to infer a second prediction based on the text input and the explanation using a large language model, to fuse the first prediction and the second prediction to generate a fused prediction, to generate a reflection based on the fused prediction, the second prediction, and the text input, and to refine the text input based on the reflection.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram showing time series prediction using a multi-modal prototype-based encoder, in accordance with an embodiment of the present invention;

FIG. 2 is a block diagram showing detail on the multi-modal prototype-based encoder, in accordance with an embodiment of the present invention;

FIG. 3 is a block/flow diagram of a method for training and using a multi-modal prototype-based encoder to perform time series prediction, in accordance with an embodiment of the present invention;

FIG. 4 is pseudo-code showing the training and use of a multi-modal prototype-based encoder to perform time series prediction, in accordance with an embodiment of the present invention;

FIG. 5 is a block diagram of a healthcare facilities that uses explainable multi-modal time series prediction to assist with medical decision making, in accordance with an embodiment of the present invention;

FIG. 6 is a computing device that includes a multi-modal prototype-based encoder to predict and respond to future time series states, in accordance with an embodiment of the present invention;

FIG. 7 is a diagram of an exemplary neural network architecture that can be used to implement part of the multi-modal prototype-based encoder, in accordance with an embodiment of the present invention; and

FIG. 8 is a diagram of an exemplary deep neural network architecture that can be used to implement part of the multi-modal prototype-based encoder, in accordance with an embodiment of the present invention.

### DETAILED DESCRIPTION OF PREFERRED EMBODIMENTS

In real-world scenarios, time series data may be influenced by external factors that may not be strictly temporal, but which are important for a comprehensive understanding of the system. The broader context of the system that originates the time series data, for example in the form of textual data or other supplementary information, therefore provides valuable insights into the underlying time series. This contextual information can help analytical models achieve accurate predictions and provide reliable and faithful explanations.

A multi-modal interpretable framework may therefore be used to predict time series, making use of a time series model with a large language model (LLM). This framework enhances predictive performance based on enriched multi-modal inputs and can render predictions with comprehensive explanations across different modalities.

These models may be applied to, for example, a treatment regime for a patient in a medical scenario, where the time series may track measurements of the patient's condition such as heart rate, blood pressure, oxygen levels, and other physiological measurements. Focusing solely on this intrinsic temporal data risks overlooking important contextual factors that could significantly influence the patient's status. Textual records, such as historical diagnoses and medications could provide valuable supplementary information that enriches the time series information.

The present embodiments leverage prototype-based learning across two modalities that provide exemplary cases, which can be comprehended by the LLM to enhance an original prediction from the time series data. The explanations that are generated based on textual prototypes can further be refined by the LLM with guidance of ground truth values, which provides more relevant information to facilitate the predictions from different components.

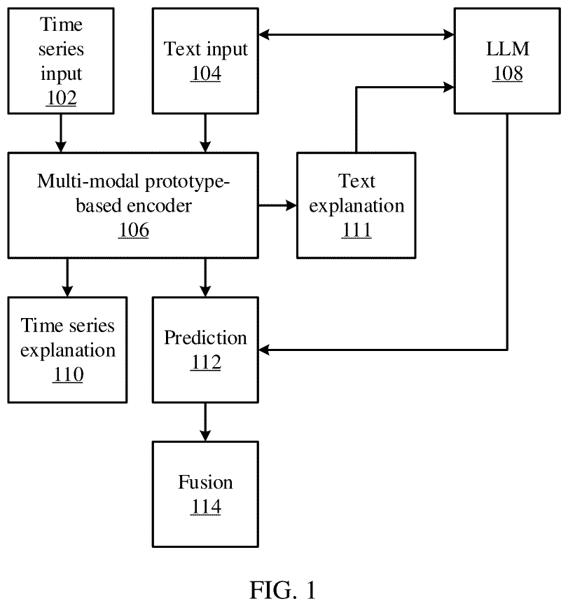

Referring now to FIG. 1, a framework for time series analysis is shown. A time series input 102 and a text input 104 are provided to a multi-modal prototype-based encoder 106, which end-to-end generates prediction 112 and further generates a time series explanation 110 and a text explanation 111 across different modalities. The encoder 106 may be implemented using a two-dimensional neural network with the kernel size and unit stride being specified as hyperparameters. The explanations 110 and 111 include comparisons to prototypes from training data (e.g., the most representative data cases).

Each multi-modal input includes a time series x=(x₁, x₂, . . . , x_{T})∈ [symbol] ^{N×T} with N variables and T time steps, and contextual text data s. Based on the historical time series and the textual context, the goal is to predict a future outcome y, either as a discrete value for classification tasks or as a continuous value for regression tasks. The encoder 106 is denoted here in as [symbol] _{enc} and provides an initial prediction 112 and case-based explanations 110 and 111.

An LLM 108 may include multiple subcomponents. One is a prediction LLM that is denoted as [symbol] ₚ and that provides an initial prediction and case-based explanation based on the understanding of context. An evaluation LLM [symbol] e generates feedback and a refinement LLM [symbol] ᵣ refines the textual context 104 based on the feedback. The predictions generated by the LLM 108 are combined with the output prediction 112 of the encoder 106 at fusion block 114 to generate the final prediction.

The reasoning and inference capabilities of LLMs on real-world time series context are exploited with three language agents interacting with the fine-tuned multi-modal prototype-based encoder 106. These agents may be implemented by a same LLM, prompted with different sets of instructions. A prediction LLM is prompted to provide classification results based on input text and corresponding prototypes. A reflection LLM is prompted to provide textual reflective feedback that identifies textual noise or inconsistencies, based on the input text, the prediction, and the ground truth label. A refinement LLM is prompted to refine the input text, based on the summarized textual reflective feedback.

The fine-tuned encoder 106 helps [symbol] ₚ render accurate predictions by enriching the text input s with case-based explanations 111. Specifically, the text explanations 111 highlight the most relevant textual segments within s with indicative class prototypes, which facilitates the language agent [symbol] ₚ to comprehend the real-world context with focus. The most relevant ω segment-prototype pairs are selected based on representation similarities:

$$exp {l}_{s} = {⋃}_{ω} ( {p}_{i}^{( c )} , {s}_{j : j + {w}^{′} - 1} )$$

$$where {⋃}_{ω} ( i , j , c ) = \{ arg {top}_{ω} ( {‖}_{i , j , c} {S}_{i , j}^{( c )} ) , ∀ i , j , c \}$$

Note that i, j, c denotes the prototype index, segment index, and class index, respectively. As explₛ can contain relevant contextual guidance across multiple classes, it augments the input space and removes semantic ambiguity for prediction agent [symbol] ₚ. Therefore, the prediction is drawn as ŷ_{LLM}= [symbol] ₚ(s, explₛ).

The final prediction is determined as a fusion of [symbol] _{enc} and [symbol] ₚ. Specifically, fusion 114 linearly combines the continuous prediction probabilities ŷ_{enc} and the discrete prediction ŷ_{LLM} as ŷ=αŷ_{enc}+(1−α)ŷ_{LLM}, where α=[0,1] is a hyperparameter selected from validation data. The encoder [symbol] _{enc} and the prediction agent [symbol] ₚ enhance each other based on their unique strengths. The encoder [symbol] _{enc} is fine-tuned based on explicit supervised signals, ensuring accuracy in capturing temporal and contextual dependencies of multi-modal time series. On the other hand, [symbol] ₚ contributes deep semantic understanding drawn from extensive text corpora. By fusing predictions from two distinct perspectives, more accurate and comprehensive predictions are possible for complex multi-modal time series.

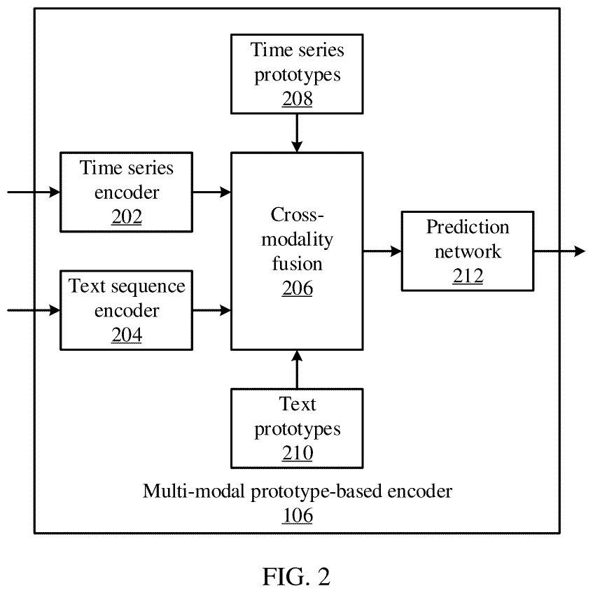

Referring now to FIG. 2, additional detail is shown for the multi-modal prototype-based encoder 106. To capture the complex temporal and semantic dependencies, a separate time series encoder 202 and text sequence encoder 204 are employed, parameterized as ε_{θ} and ε_{φ} respectively. For a time series input 102, the time series encoder 202 maps the entire sequence into one or multiple representations, which serve as candidates for prototype learning. The encoder makes use of time series prototypes 208 and text prototypes 210, with the cross-modality fusion 206 combining the encoders' outputs with consideration of the prototypes to generate an output that a non-negative, fully connected prediction network 212 uses to generate the final predictions of the encoder 106.

Time series prototypes 208 may be representative or exemplar time series time series segments from the training dataset and text prototypes 210 may be representative or exemplar textual segments (e.g., phases) from the training dataset. During comparison, similarity scores are determined between the inputs and prototypes, with each prototype having a respective fixed weight in the prediction network 212 after training. The score and weight both contribute to the final contribution, which is interpretable.

For text input 104, before being encoded by text sequence encoder 204, it may first be processed by a frozen, pre-trained language model into a sequence embedding as eₛ=PLM(s)∈ [symbol] ^{d}^{ₛ}^{×L}, with dₛ being the dimensionality and with L being the number of tokens or sentences. Alternatively, the pre-trained language model may be selected to cope with different scales of text length, for example using a bidirectional encoder representations from transformers (BERT) approach for shorter text inputs (e.g., fewer than 300 words) to model text embedding at the token level, and using sentence transformers to embed text at the phrase- or sentence-level, which leads to different granularities of text explanations.

The choice of ε_{θ} and ε_{φ} also affects the granularity of explanations. The prototypes are learned based on sequence representations and will associate with the counterparts in the input space, where the correspondences are determined by the encoders. Convolution-based encoders may be used for both modalities to capture the fine-grained sub-sequence patterns. Given ε_{θ} with h kernels of kernel size w, and given ε_{φ} with h′ kernels of kernel size w′, the multi-modal encoding may be represented as:

$${Z}_{time} = ( {z}_{1} , … , {z}_{T - w + 1} ) = {ℰ}_{θ} ( x )$$

$${Z}_{text} = ( {z}_{1}^{′} , … , {z}_{T - w + 1}^{′} ) = {ℰ}_{ϕ} ( {e}_{s} )$$

where zᵢ∈ [symbol] ^{h} and zᵢ′∈ [symbol] ^{h′} denote the representation for the i^{th} time series segment and j^{th} text segment, respectively. One or multiple kernel sizes may be used for each modality to enrich the explanation space toward more comprehensive and accurate predictive analysis.

To cope with potential data imbalances from real-world multi-modal time series and to ensure robust explanations, prototypes may be allocated for each class. For a class c∈(1, . . . , C)=[C], k time series prototypes are initialized as

$${P}_{time}^{( c )} = ( {p}_{1}^{( c )} , … , {p}_{k}^{( c )} ) = {ℝ}^{k \times h}$$

and k′ text prototypes are initialized as

$${P}_{text}^{( c )} = ( {p}_{1}^{′ ( c )} , … , {p}_{{k}^{′}}^{′ ( c )} ) = {ℝ}^{{k}^{′} \times {h}^{′}}$$

for training parameters that have the same dimensionality as the corresponding segment representations. This provides

$${P}_{time} = [ {P}_{time}^{( 1 )} , … , {P}_{time}^{( C )} ] ∈ {ℝ}^{C \cdot k \times h} and {P}_{text} = [ {P}_{text}^{( 1 )} , … , {P}_{text}^{( c )} ] ∈ {ℝ}^{C \cdot {k}^{′} \times {h}^{′}} .$$

The prototypes are associated with training segments once learning objectives converge.

During the forward pass, each segment representation and prototype from its own modality are compared, where the similarity score is evaluated as an exponential function based on their L2 distance, denoted as K(.,.). Moreover, the score between each prototype and the most pertinent segment will be preserved to represent the prototype-input similarity. For example, given a time series prototype

$${p}_{i}^{( c )}$$

and segment representations Zₜᵢₘₑ, the similarity score

$${S}_{i}^{( c )}$$

is calculated as:

$${S}_{i , j}^{( c )} = K ( {p}_{i}^{( c )} , {z}_{j} ) = exp ( - {‖ {p}_{i}^{( c )} - {z}_{j} ‖}_{2}^{2} ) ∈ [ 0 , 1 ] ,$$

$${S}_{i}^{( c )} = max ( {S}_{i , 1}^{( c )} , … , {S}_{i , T - w + 1}^{( c )} )$$

Similarity scores are concatenated across all class prototypes for each modality to obtain

$${S}_{time} = {‖}_{i , c} {S}_{i}^{( c )} ∈ {ℝ}^{k \cdot C} and {S}_{text} = {‖}_{i , c} {S}_{i}^{′ ( c )} ∈ {ℝ}^{{k}^{′} \cdot C}$$

that contains the temporal and contextual relevance of the multi-modal input. Finally, the cross-modal relevance are fused toward the final prediction. To quantify the contribution of the relevance represented by each prototype to each class, a weight matrix W∈ [symbol] ^{C×(k+k′)} is used with the non-negative constraint on each entry. As such, the prediction probabilities are computed as ŷ_{enc}=Softmax(W·[Sₜᵢₘₑ∥Sₜₑₓₜ]).

The learning objectives include three regularization terms that reinforce the interpretability of multi-modal prototypes. The basic objective is the cross-entropy loss for the prediction drawn from multi-modal explainable artifacts [symbol] _{CE}=Σ_{x,s,y}ylog(ŷ_{enc})+(1−y)log(1−ŷ_{enc}). A clustering structure of segments is encouraged in the representation space by enforcing each segment representation to be adjacent to its closest prototype. Each prototype is regularized to be as close to a segment representation as possible, so as to help the prototype locate the most evidencing segment. The regularization terms are denoted as [symbol] _{c} and [symbol] ₑ respectively, where the modality and class notations are omitted for the ease of understanding:

$${ℒ}_{c} = {\sum }_{{z}_{j} ∈ {Z}_{( \cdot )}} {min}_{{p}_{i} ∈ {P}_{( \cdot )}} {‖ {z}_{j} - {p}_{i} ‖}_{2}^{2} ,$$

$${ℒ}_{e} = {\sum }_{{p}_{i} ∈ {P}_{( \cdot )}} {min}_{{z}_{j} ∈ {Z}_{( \cdot )}} {‖ {p}_{i} - {z}_{j} ‖}_{2}^{2}$$

Moreover, a diverse structure of prototype representations is encouraged to maintain a compact explanation space, which penalizes their similarities via a hinge loss C_{d}, with a threshold dₘᵢₙ:

$${ℒ}_{d} = {\sum }_{i = 1} {\sum }_{j \neq i} max ( 0 , {d}_{min} - {‖ {p}_{i} - {p}_{j} ‖}_{2}^{2} )$$

The full objective is written as: [symbol] = [symbol] _{CE}+λ₁ [symbol] _{c}+λ₂ [symbol] ₑ+λ₃ [symbol] _{d}, with hyperparameters λ₁, λ₂, and λ₃ that balance learning components towards an optimal explainable prediction.

After learning objectives converge, the multi-modal prototypes are well-regularized that reflect good explanation properties. However, these prototypes are still not readily explainable as they are only close to some exemplar segments in the representation space. Therefore, prototype projection is performed to associate each prototype with a training segment from its own class that preserves [symbol] ₑ in the representation space:

$${p}_{i}^{( c )} ← {arg min}_{{z}_{j} ∈ {Z}_{( \cdot )}^{( c )}} {‖ {p}_{i}^{( c )} - {z}_{i} ‖}_{2}^{2} , ∀ {p}_{i}^{( c )} ∈ {P}_{( \cdot )}^{( c )}$$

By associating each prototype with a training segment in the representation space, the multi-modal physical meaning is induced. During testing phase, a multi-modal instance will be compared with prototypes across different modalities to infer predictions, where the similarity scores, contribution weights, and prototypes' class information assemble the explanation artifacts for reasoning.

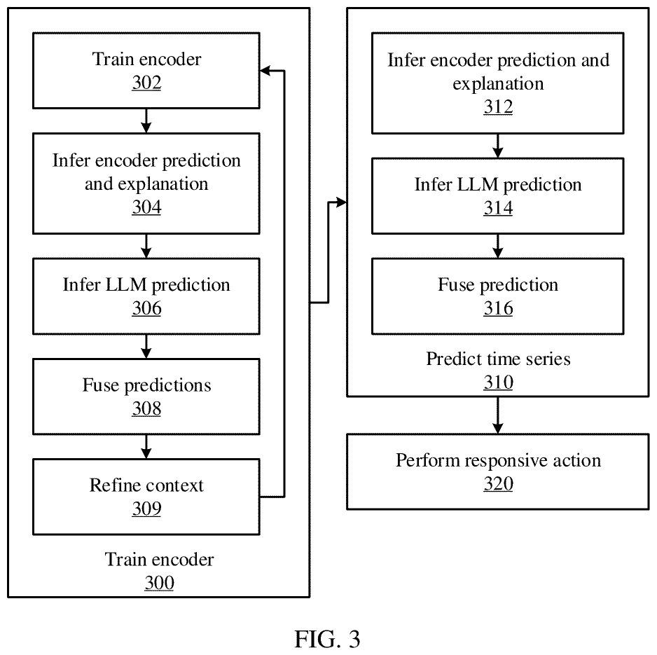

Referring now to FIG. 3, a method of training and using a time series prediction model is shown. Block 300 trains the multi-modal prototype-based encoder, block 310 uses the trained encoder and a pretrained LLM to predict time series information using textual context, and block 320 performs an action responsive to the prediction.

The training 300 includes training 302 the encoder model [symbol] _{enc} based on multi-modal data, including time series input 102 and text input 104. In this case, the time series input 102 and the text input 104 are training samples from a training dataset and are accompanied by ground truth values for the prediction. Block 304 infers a prediction and explanation using the encoder 106 and block 306 infers a prediction using the LLM 108. Block 308 fuses these two predictions together to come to a final prediction. Block 309 refines the context by generating a reflection and then refining the textual context based on the reflection. This process continues for some predetermined maximum number of iterations.

During testing, block 310 accepts a new input time series 102 and a new text input 104 and block 312 infers the encoder prediction and explanation using the trained encoder 106. Block 314 infers the LLM prediction using the LLM 108 and block 316 fuses the encoder prediction and the LLM prediction to generate an output prediction. This output prediction may predict future measurements from sensors generating the time series and may further identify a state of the system using, e.g., a classifier.

Based on this prediction, block 320 performs a responsive action. In the example of a medical scenario, where the time series input 102 and the text input 104 relate to a medical condition of a patient, the prediction may indicate future health states (e.g., heart rate or blood pressure) or may identify a label for a health condition of the patient, such as a diagnosis of a disease. The responsive action 320 may then include a treatment action that is selected responsive to the health state or health condition. In some cases the responsive action may include generating a report for medical professionals to assist in medical decision making. In some cases the responsive action may include the automatic administration or halting of a treatment, such as by intravenous administration of a medication.

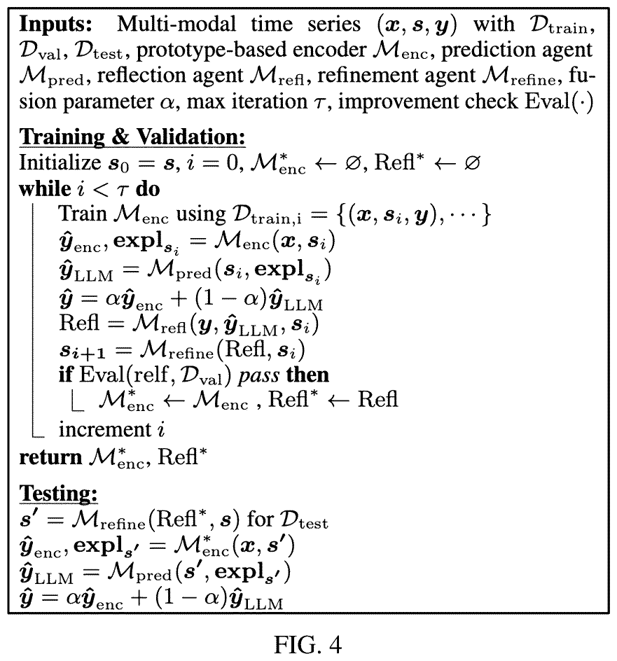

Referring now to FIG. 4, pseudo-code is shown for the training and use of a model for explainable multi-modal time series prediction. While the prediction agent [symbol] _{pred} leverages explainable artifacts to make informed predictions, it is not inherently designed to fit the context of multi-modal time series data, which can lead to inaccurate predictions when the quality of the textual content is inferior. Two additional language agents, [symbol] _{refl} and [symbol] _{refine} may be used to generate reflective feedback and refinements on the context, respectively, for better predictive insights. Reflection, as used herein, refers to the process of identifying textual noise or inconsistencies by comparing the prediction to the ground truth label. If the prediction is correct, then the reflection may highlight important phrases that contribute to the prediction. If the prediction is incorrect, then the reflection may identify phrases which lead to the incorrect prediction. The reflection output may include natural language text. Refinements may be generated by letting the refinement LLM read and understand the reflection and input text.

Given the prediction ŷ_{LLM} generated by the prediction agent [symbol] _{pred}, representing semantic trends suggested by the text prototypes, the reflection agent [symbol] _{refl} aims to understand the reasoning behind the implicit prediction logic of [symbol] _{pred}. Based on the input text s and the prediction ŷ_{LLM}, against a ground truth y, an indicative reflection is obtained as feedback Refl from [symbol] _{refl}, to provide actionable insights for refinement. Guided by the feedback, [symbol] _{refine} refines the previous text s into s+1 by selecting and emphasizing the most relevant content, ensuring that important patterns are appropriately contextualized.

The refinement is integrated via reflection into the optimization loop during training 300. Once the textual context is improved, it is used to retrain the multi-modal prototype-based encoder [symbol] _{enc} for the next iteration. As such, the explanation (shown in the quality of the prototypes) and predictive performance of [symbol] _{enc} is improved through the iterative process. The prediction agent [symbol] _{enc} yields better prediction with more informative inputs, further enhancing the accuracy of ŷ. The trajectory of the predictive performance is evaluated and the iterations are terminated if no improvement is observed or if a maximum number of iterations is reached.

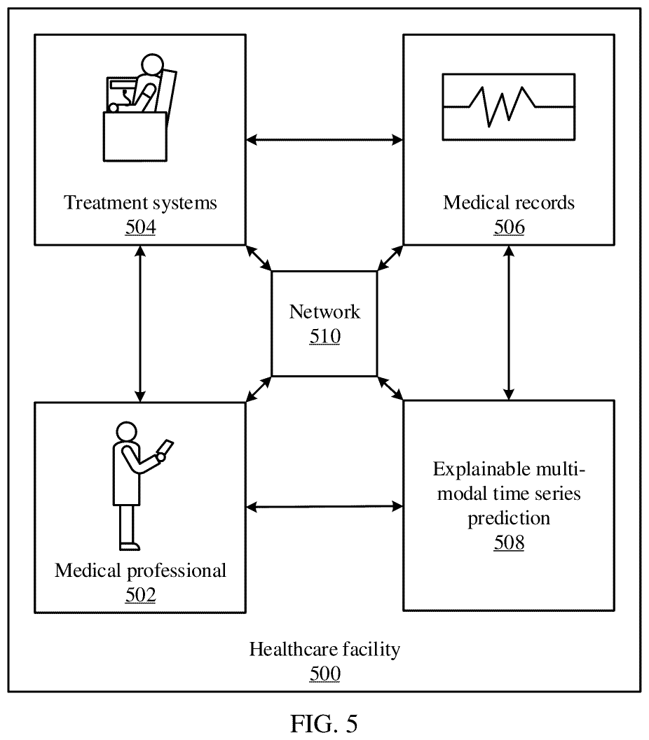

Referring now to FIG. 5, a diagram of RAG-based solutions to health issues is shown in the context of a healthcare facility 500. Explainable multi-modal time series prediction 508 may be used to predict a future health state or generate a diagnosis for a patient responsive to time series data and contextual text information, for example based on the patient's medical records 506 and general information relating to medical conditions.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the explainable multi-modal time series prediction 508, the medical professionals 502 may then make medical decisions about patient healthcare suited to the patient's needs. For example, the medical professionals 502 may make treatment decisions based on a diagnosis generated by the explainable multi-modal time series prediction 508 and may prescribe particular medications, surgeries, and/or therapies that are appropriate to the diagnosis disease.

The different elements of the healthcare facility 500 may communicate with one another via a network 510, for example using any appropriate wired or wireless communications protocol and medium. Thus explainable multi-modal time series prediction 508 receives data from treatment systems 504, medical professionals 502, and from medical records 506, and predicts future time series measurements and labels. The explainable multi-modal time series prediction 508 may further coordinate with treatment systems 504 in some cases to automatically administer or alter a treatment. For example, if the solution indicates a particular treatment, the system may automatically trigger implement the treatment, such as by initiating or halting the administration of a medication.

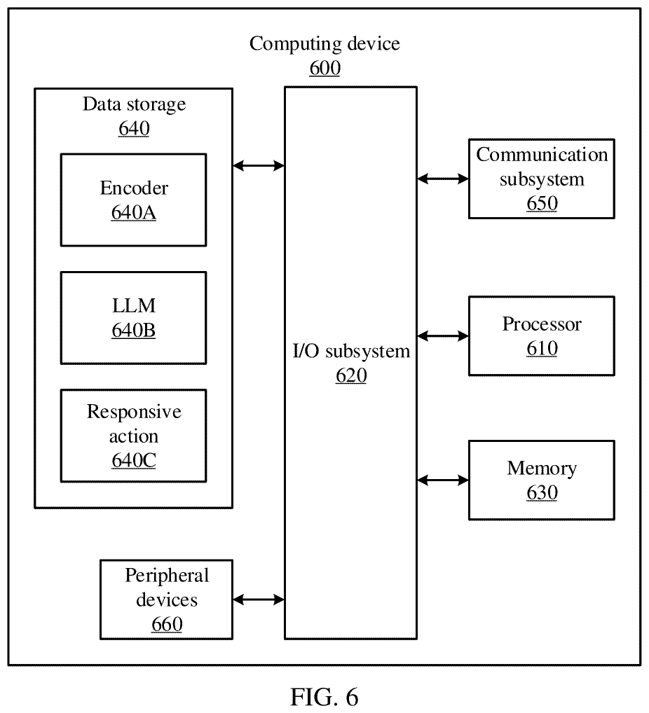

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for implementing the encoder, 640B for implementing the LLM, and/or 640C for performing responsive actions. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

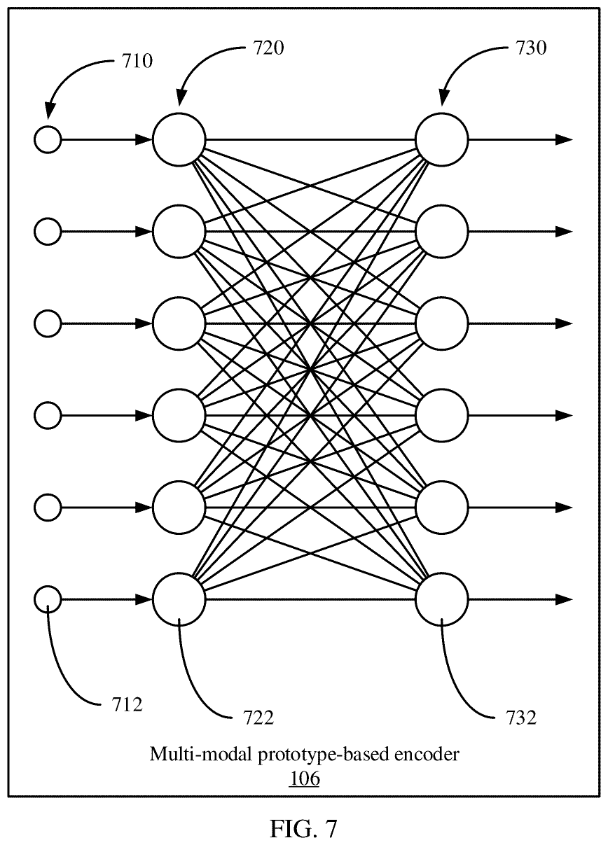

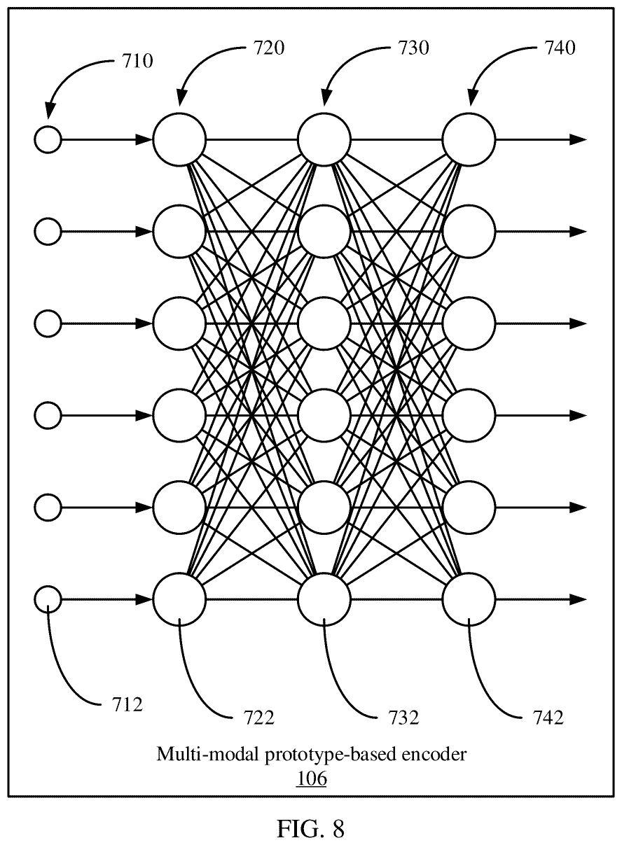

Referring now to FIGS. 7 and 8, exemplary neural network architectures are shown, which may be used to implement parts of the present machine learning models, such as the multi-modal prototype-based encoder 106. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
inferring a first prediction and an explanation based on a time series input and a text input using a multi-modal prototype-based encoder;
inferring a second prediction based on the text input and the explanation using a large language model;
fusing the first prediction and the second prediction to generate a fused prediction;
generating a reflection based on the fused prediction, the second prediction, and the text input; and
refining the text input based on the reflection.

2. The method of claim 1, further comprising iteratively training the multi-modal prototype-based encoder, including generating the reflection and refining the text input for new training samples at each iteration.

3. The method of claim 2, wherein iteratively training the multi-modal prototype-based encoder returns a trained encoder, the reflection from a final iteration, and the refined text input from the final iteration.

4. The method of claim 3, further comprising:
refining a new text input using the reflection from the final iteration;
inferring a new first prediction and a new explanation based on a new time series input and the refined new text input using the trained encoder;
inferring a new second prediction based on the refined new text input and the new explanation using the large language model;
fusing the new first prediction and the new second prediction to generate a new fused prediction; and
performing an action responsive to the fused prediction.

5. The method of claim 4, wherein the new time series input and the new text input relate to a health condition of a patient and wherein the new fused prediction identifies a future health condition of the patient.

6. The method of claim 5, wherein the action reports the future health condition of the patient to a medical professional to assist in medical decision making.

7. The method of claim 5, wherein the action performs an automated treatment action to affect the future health condition of the patient.

8. The method of claim 2, wherein iteratively training the multi-modal prototype-based encoder includes a loss function that includes a cross-entropy loss term, clustering regularization terms, and a diversity regularization term.

9. The method of claim 1, wherein the multi-modal prototype-based encoder is implemented as a machine learning model.

10. The method of claim 1, wherein the multi-modal prototype-based encoder encodes the time series input and the text input using respective encoders and aligns them with respective prototypes.

11. A system, comprising:
a hardware processor; and
a memory that stores a computer program which, when executed by the hardware processor, causes the hardware processor to:
infer a first prediction and an explanation based on a time series input and a text input using a multi-modal prototype-based encoder;
infer a second prediction based on the text input and the explanation using a large language model;
fuse the first prediction and the second prediction to generate a fused prediction;
generate a reflection based on the fused prediction, the second prediction, and the text input; and
refine the text input based on the reflection.

12. The system of claim 11, wherein the computer program further causes the hardware processor to iteratively train of the multi-modal prototype-based encoder, including generation of the reflection and refinement of the text input for new training samples at each iteration.

13. The system of claim 12, wherein the iterative training of the multi-modal prototype-based encoder returns a trained encoder, the reflection from a final iteration, and the refined text input from the final iteration.

14. The system of claim 13, wherein the computer program further causes the hardware processor to:
refine a new text input using the reflection from the final iteration;
infer a new first prediction and a new explanation based on a new time series input and the refined new text input using the trained encoder;
infer a new second prediction based on the refined new text input and the new explanation using the large language model;
fuse the new first prediction and the new second prediction to generate a new fused prediction; and
perform an action responsive to the fused prediction.

15. The system of claim 14, wherein the new time series input and the new text input relate to a health condition of a patient and wherein the new fused prediction identifies a future health condition of the patient.

16. The system of claim 15, wherein the action reports the future health condition of the patient to a medical professional to assist in medical decision making.

17. The system of claim 15, wherein the action performs an automated treatment action to affect the future health condition of the patient.

18. The system of claim 12, wherein the iterative training of the multi-modal prototype-based encoder includes a loss function that includes a cross-entropy loss term, clustering regularization terms, and a diversity regularization term.

19. The system of claim 11, wherein the multi-modal prototype-based encoder is implemented as a machine learning model.

20. The system of claim 11, wherein the multi-modal prototype-based encoder encodes the time series input and the text input using respective encoders and aligns them with respective prototypes.
